(19) United States
(12) Patent Application Publication
(10) Pub. No.: US 2025/0156499 A1
(43) Pub. Date: May 15, 2025

(54) DATA PROCESSING METHOD AND APPARATUS, ELECTRONIC DEVICE, AND STORAGE MEDIUM

(71) Applicant: XIAMEN INDUSTRIAL TECHNOLOGY RESEARCH INSTITUTE CO., LTD., Xiamen (CN)

(72) Inventors: Yong ZHANG, Xiamen (CN); Huayu OUYANG, Xiamen (CN); Guozhong CAO, Xiamen (CN)

(73) Assignee: XIAMEN INDUSTRIAL TECHNOLOGY RESEARCH INSTITUTE CO., LTD., Xiamen (CN)

(21) Appl. No.: 19/021,304

(22) Filed: Jan. 15, 2025

Related U.S. Application Data
(63) Continuation of application No. PCT/CN2024/115924, filed on Aug. 30, 2024.

(30) Foreign Application Priority Data
Sep. 5, 2023 (CN) 202311136280.4

(51) Int. Cl. G06F 17/16; G06F 7/544; H03M 1/12

(52) U.S. Cl. CPC G06F 17/16; G06F 7/5443; H03M 1/1245

(57) ABSTRACT

A data processing method and apparatus, an electronic device, and a storage medium are provided. The method includes: obtaining to-be-processed array data; performing first mapping on the to-be-processed array data to obtain target array data, and determining a weight corresponding to each target data in the target array data during the first mapping; performing multiplier and accumulation operation on the target data according to a first or second rule to obtain a first and second operation values; performing analog-to-digital conversion on the first operation value and the second operation value to obtain a first quantized value and a second quantized value, respectively; performing second mapping on the first quantized value and the second quantized value based on the weights to obtain a third quantized value and a fourth quantized value; and summing the third quantized value and the fourth quantized value to obtain a target output value.

### CROSS REFERENCE TO THE RELATED APPLICATIONS

The present application is a continuation application of International Application No. PCT/CN2024/115924, filed on Aug. 30, 2024, which is based upon and claims priority to Chinese Patent Application No. 202311136280.4, filed on Sep. 5, 2023, the entire contents of which are incorporated herein by reference.

### TECHNICAL FIELD

The present application relates to the field of information technologies, and in particular to a data processing method and apparatus, an electronic device, and a storage medium.

### BACKGROUND

The architecture of a conventional computing in memory (CIM) circuit is as follows: to-be-processed array data is transferred to an array circuit through a peripheral circuit for multiplier and accumulation (MAC) operation to obtain a randomly distributed and widely ranging MAC current or voltage value, and then the MAC current or voltage value is converted into a corresponding digital value by using a high dynamic range sampling analog-to-digital converter (ADC) circuit. Due to the random distribution and wide range of the to-be-processed array data, such a circuit architecture will lead to high power consumption of the array circuit, has a significant impact on the sampling precision and conversion time of the ADC, and is not conducive to improving the energy efficiency of the CIM circuit.

### SUMMARY

In view of this, embodiments of the present application provide a data processing method and apparatus, an electronic device, and a storage medium to at least solve the above technical problems existing in the related art.

According to a first aspect of the present application, an embodiment of the present application provides a data processing method, which includes:
obtaining to-be-processed array data;
performing first mapping on the to-be-processed array data to obtain target array data, and determining a weight corresponding to each target data in the target array data during the first mapping; where a numerical range of the target data in the target array data is less than a numerical range of data in the to-be-processed array data;
performing multiplier and accumulation operation on the target data with the weight of 1 according to a first rule to obtain a first operation value; and performing the multiplier and accumulation operation on the target data with the weight not equal to 1 according to a second rule of multiplying a same column by a same value to obtain a second operation value;
performing analog-to-digital conversion on the first operation value and the second operation value to obtain a first quantized value and a second quantized value, respectively;
performing second mapping on the first quantized value and the second quantized value based on the weights to obtain a third quantized value and a fourth quantized value; where the second mapping is reverse to the first mapping; and
summing the third quantized value and the fourth quantized value to obtain a target output value.

Optionally, the step of performing the first mapping on the to-be-processed array data to obtain the target array data, and determining the weight corresponding to each target data in the target array data during the first mapping, the numerical range of the target data in the target array data being less than the numerical range of the data in the to-be-processed array data, may include:
determining a numerical range corresponding to each data in the to-be-processed array data; and
performing the first mapping on each data based on the numerical range corresponding to each data to maintain the data belonging to a first numerical range unchanged and map the data greater than the first numerical range to data of the first numerical range to obtain the target array data, and determining a weight of each data during the first mapping as the weight corresponding to each target data in the target array data during the first mapping.

Optionally, the step of performing the first mapping on each data based on the numerical range corresponding to each data to maintain the data belonging to the first numerical range unchanged and map the data greater than the first numerical range to the data of the first numerical range to obtain the target array data, and determining the weight of each data during the first mapping as the weight corresponding to each target data in the target array data during the first mapping may include:
when the data belongs to the first numerical range, determining that the data is maintained unchanged during the first mapping and the weight corresponding to the target data obtained after the first mapping on the data is 1;
when the data belongs to a second numerical range, determining that the data is reduced by a first preset multiple during the first mapping and the weight corresponding to the target data obtained after the first mapping on the data is the first preset multiple; where the second numerical range is greater than the first numerical range; and
when the data belongs to a third numerical range, determining that the data is reduced by a second preset multiple during the first mapping and the weight corresponding to the target data obtained after the first mapping on the data is the second preset multiple; where the third numerical range is greater than the second numerical range, and the second preset multiple is greater than the first preset multiple.

Optionally, the step of performing the analog-to-digital conversion on the first operation value and the second operation value to obtain the first quantized value and the second quantized value respectively may include:
calculating a first difference between the first operation value and a reference value and a second difference between the second operation value and the reference value by an analog-to-digital converter, respectively;
when absolute values of the first and second differences are less than a threshold, sampling analog signals output from different bits of the analog-to-digital converter in order from low to high bits; and
determining the first quantized value based on a bit value of the analog-to-digital converter corresponding to the analog signal matching the first operation value, and determining the second quantized value based on a bit value of the analog-to-digital converter corresponding to the analog signal matching the second operation value.

Optionally, the step of calculating the second difference between the second operation value and the reference value by the analog-to-digital converter may include:
determining a correction value corresponding to the second operation value by the analog-to-digital converter based on the weight of the target data corresponding to the second operation value;
calculating a third difference between the second operation value and the correction value; and
designating a difference between the third difference and the reference value as the second difference between the second operation value and the reference value.

Optionally, the step of performing the second mapping on the first quantized value and the second quantized value based on the weights to obtain the third quantized value and the fourth quantized value, the second mapping being reverse to the first mapping, may include:
multiplying the weight of the target data corresponding to the first quantized value by the first quantized value to obtain the third quantized value; and
multiplying the weight of the target data corresponding to the second quantized value by the second quantized value to obtain the fourth quantized value.

According to a second aspect of the present application, an embodiment of the present application provides a data processing apparatus, which includes:
an obtaining module, configured to obtain to-be-processed array data;
a first mapping module, configured to perform first mapping on the to-be-processed array data to obtain target array data, and determine a weight corresponding to each target data in the target array data during the first mapping; where a numerical range of the target data in the target array data is less than a numerical range of data in the to-be-processed array data;
an operation module, configured to perform multiplier and accumulation operation on the target data with the weight of 1 according to a first rule to obtain a first operation value; and perform the multiplier and accumulation operation on the target data with the weight not equal to 1 according to a second rule of multiplying a same column by a same value to obtain a second operation value;
an analog-to-digital conversion module, configured to perform analog-to-digital conversion on the first operation value and the second operation value to obtain a first quantized value and a second quantized value, respectively;
a second mapping module, configured to perform second mapping on the first quantized value and the second quantized value based on the weights to obtain a third quantized value and a fourth quantized value; where the second mapping is reverse to the first mapping; and
a summation module, configured to sum the third quantized value and the fourth quantized value to obtain a target output value.

Optionally, the first mapping module may be configured to:
determine a numerical range corresponding to each data in the to-be-processed array data; and
perform the first mapping on each data based on the numerical range corresponding to each data to maintain the data belonging to a first numerical range unchanged and map the data greater than the first numerical range to data of the first numerical range to obtain the target array data; and determine a weight of each data during the first mapping as the weight corresponding to each target data in the target array data during the first mapping.

According to a third aspect of the present application, an embodiment of the present application provides an electronic device, which includes:
at least one processor; and
a memory in communication connection with the at least one processor;
where the memory stores instructions executable by the at least one processor, and the instructions are executed by the at least one processor to cause the at least one processor to execute the data processing method according to the first aspect or any implementation of the first aspect.

According to a fourth aspect of the present application, an embodiment of the present application provides a computer-readable storage medium having stored therein computer instructions that, when executed by a computer, cause the computer to perform the data processing method according to the first aspect or any implementation of the first aspect.

According to the data processing method and apparatus, the electronic device, and the storage medium provided in the embodiments of the present application, the first mapping is performed on the to-be-processed array data to obtain the target array data, and the weight corresponding to each target data in the target array data during the first mapping is determined; where the numerical range of the target data in the target array data is less than that of the data in the to-be-processed array data. As such, the numerical range of the to-be-processed array data can be effectively converged, making the input data of the multiplier and accumulation operation process (that is, the numerical range of the target array data) smaller, thereby reducing computational power consumption of array circuits. In addition, the multiplier and accumulation operation is performed on the target data with the weight of 1 according to the first rule to obtain the first operation value, and the multiplier and accumulation operation is performed on the target data with the weight not equal to 1 according to the second rule of multiplying the same column by the same value to obtain the second operation value, thereby the second operation value can be maintained linear, and the first operation value maintains conversion accuracy. On the one hand, the conversion accuracy loss of overall operation values is reduced, and on the other hand, the second quantized value subsequently corresponding to the second operation value can be restored. Then, the analog-to-digital conversion is performed on the first operation value and the second operation value separately to obtain the first quantized value and the second quantized value, and the first operation value and the second operation value have smaller numerical ranges, which can improve the conversion precision and time of an analog-to-digital conversion circuit. Next, the second mapping is performed on the first quantized value and the second quantized value based on the weights to obtain the third quantized value and the fourth quantized value, and the second mapping is reverse to the first mapping, thereby achieving the restoration of the second quantized value corresponding to the second operation value. Finally, the third quantized value and the fourth quantized value are summed to obtain the target output value, thereby achieving the restoration of the output data.

The above description is merely an overview for the technical solutions of the present application. In order to have a clearer understanding for the technical means of the present application to implement in accordance with the contents of the specification, and in order to make the above and other purposes, features and advantages of the present application more obvious and understandable, specific implementations of the present application are specially described below.

### BRIEF DESCRIPTION OF THE DRAWINGS

In order to describe the technical solutions in embodiments of the present application more clearly, the accompanying drawings required to be used in the embodiments will be simply introduced below. Apparently, the accompanying drawings described below show only some embodiments of the present application, and other drawings can further be derived by those of ordinary skill in the art according to the accompanying drawings without any creative effort.

FIG. 1 is a flowchart of a data processing method in an embodiment of the present application.

FIG. 2 is a flowchart of first mapping in an embodiment of the present application.

FIG. 3 is a flowchart of multiplier and accumulation operation on target data in an embodiment of the present application.

FIG. 4 is a flowchart of second mapping in an embodiment of the present application.

FIG. 5 is a schematic diagram of an analog-to-digital conversion process in an embodiment of the present application.

FIG. 6 is a structural diagram of a data processing apparatus in an embodiment of the present application.

FIG. 7 is a schematic diagram of a hardware structure of an electronic device in an embodiment of the present application.

### DETAILED DESCRIPTION OF THE EMBODIMENTS

In order to make the objectives, technical solutions and advantages of embodiments of the present application clearer, the technical solutions in the embodiments of the present application will be described clearly and completely below in conjunction with the accompanying drawings in the embodiments of the present application. Apparently, the described embodiments are merely a part rather than all of the embodiments of the present application. All other embodiments obtained by those skilled in the art without creative efforts based on the embodiments of the present application shall fall within the protection scope of the present application.

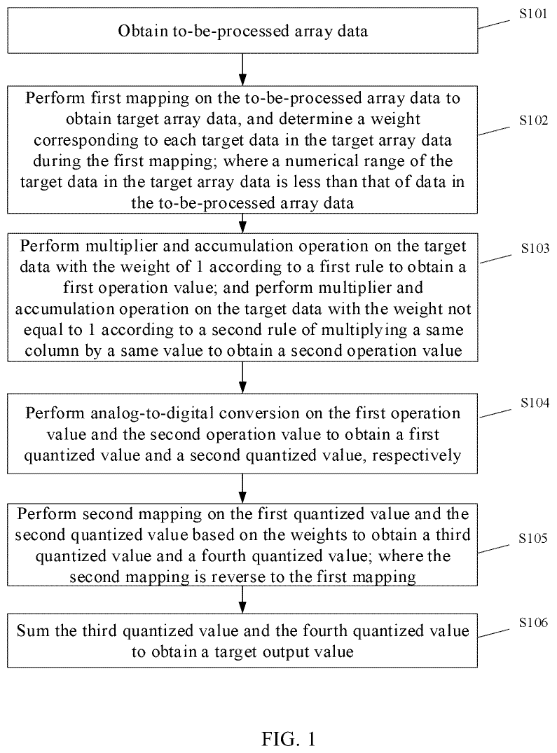

An embodiment of the present application provides a data processing method, as shown in FIG. 1, which includes:
S101, obtaining to-be-processed array data; and
S102, performing first mapping on the to-be-processed array data to obtain target array data, and determine a weight corresponding to each target data in the target array data during the first mapping; where a numerical range of the target data in the target array data is less than that of data in the to-be-processed array data.

In the embodiment, the first mapping mainly reduces the numerical range of the to-be-processed array data to obtain the target array data with a smaller numerical range.

In some embodiments, the performing the first mapping on the to-be-processed array data to obtain the target array data, and determining the weight corresponding to each target data in the target array data during the first mapping, the numerical range of the target data in the target array data being less than that of the data in the to-be-processed array data includes:
S1021, determining a numerical range corresponding to each data in the to-be-processed array data; and
S1022, performing the first mapping on each data based on the numerical range corresponding to each data to maintain the data belonging to a first numerical range unchanged and map the data greater than the first numerical range to data of the first numerical range to obtain the target array data, and determining a weight of each data during the first mapping as the weight corresponding to each target data in the target array data during the first mapping.

In some implementations, for step S1021, each data in the to-be-processed array data can be compared with an upper limit and a lower limit of each numerical range to determine the numerical range corresponding to each data.

For step S1022, the performing the first mapping on each data based on the numerical range corresponding to each data to maintain the data belonging to the first numerical range unchanged and map the data greater than the first numerical range to the data of the first numerical range to obtain the target array data, and determining the weight of each data during the first mapping as the weight corresponding to each target data in the target array data during the first mapping may include:
when the data belongs to the first numerical range, determining that the data is maintained unchanged during the first mapping and the weight corresponding to the target data obtained after the first mapping on the data is 1;
when the data belongs to a second numerical range, determining that the data is
reduced by a first preset multiple during the first mapping, the weight corresponding to the target data obtained after the first mapping on the data is the first preset multiple; and the target data obtained by reducing the data by the first preset multiple belongs to the first numerical range, that is, the data is mapped to the data of the first numerical range; the second numerical range being greater than the first numerical range refers to: a lower limit of the second numerical range is greater than or equal to an upper limit of the first numerical range; for example, the lower limit of the second numerical range is equal to the upper limit of the first numerical range, the first numerical range is less than or equal to 5, and the second numerical range is greater than 5 and less than 7; the lower limit of the second numerical range is greater than the upper limit of the first numerical range, the first numerical range is less than or equal to 5, and the second numerical range is greater than 6 and less than 7; and
when the data belongs to a third numerical range, determining that the data is reduced by a second preset multiple during the first mapping, the weight corresponding to the target data obtained after the first mapping on the data is the second preset multiple; and the target data obtained by reducing the data by the second preset multiple belongs to the first numerical range, that is, the data is mapped to the data of the first numerical range; the third numerical range being greater than the second numerical range refers to: a lower limit of the third numerical range is greater than or equal to an upper limit of the second numerical range. The third numerical range is greater than the second numerical range, in order to map data belonging to the third numerical range to the first numerical range, the second preset multiple needs to be greater than the first preset multiple.

Thus, when the data belongs to the first numerical range, the first mapping corresponding to the data is maintaining the data unchanged, which can also be understood as reducing the data by a multiple of 1; when the data is greater than the first numerical range (belongs to the second numerical range or the third numerical range), the first mapping corresponding to the data is reducing the data by a preset multiple; and the preset multiple for corresponding reduction can be determined based on the second numerical range or the third numerical range, so that the data belongs to the first numerical range after being reduced by the preset multiple.

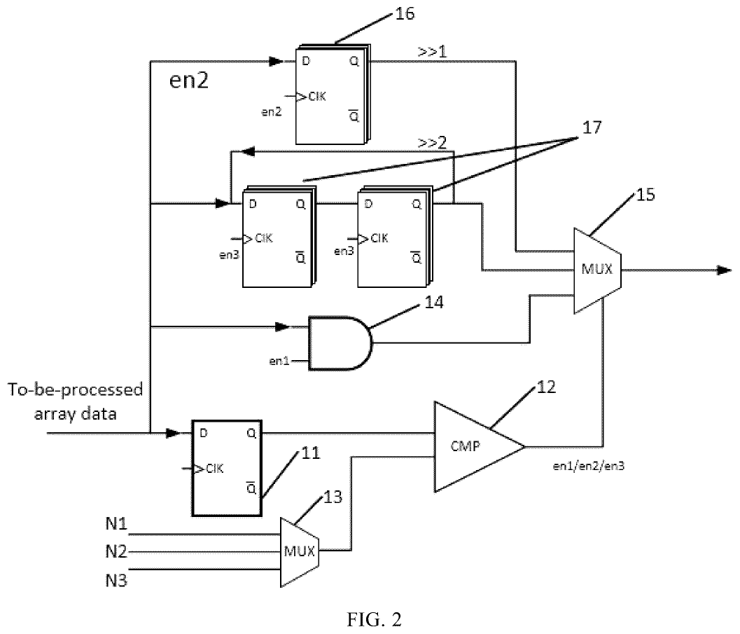

During specific implementation, a first mapping module shown in FIG. 2 can be used to perform the first mapping on the to-be-processed array data. Firstly, the to-be-processed array data is input to a comparator 12 through an input submodule 11, and preset values N1, N2, and N3 are input to the comparator (CMP) 12 through a selector switch (MUX) 13, where N1<N2<N3. Then, the comparator 12 compares the magnitude of each data in the to-be-processed array data with N1, N2, and N3.

For example, data is less than N1. It can be considered that the data belongs to the first numerical range (less than or equal to N1). A first enable signal en1 is generated, the data is transmitted to a selector switch 15 through a switch 14, and the selector switch 15 outputs the data transmitted from the switch 14 based on the first enable signal en1. As such, the data is maintained unchanged after the first mapping, that is, the reducing multiple is 1, and the corresponding weight of the target data corresponding to the data is 1.

For another example, data is greater than N1 and less than N2. It can be considered that the data belongs to the second numerical range (greater than N1 and less than N2). A second enable signal en2 is generated, the data is transmitted to the selector switch 15 through a shifter (CLK) 16, the shifter 16 can shift the data to the right by 1 bit, that is, the data is reduced by a multiple of 2, and the obtained target data is less than or equal to N1. The selector switch 15 outputs the data transmitted from the shifter 16 based on the second enable signal en2. As such, the data is reduced by a multiple of 2 after the first mapping, and the corresponding weight of the target data corresponding to the data is 2.

For example, data is greater than N2 and less than N3. It can be considered that the data belongs to the third numerical range (greater than or equal to N2 and less than or equal to N3). A third enable signal en3 is generated, the data is transmitted to the selector switch 15 through shifters 17 (including two shifters), the shifters 17 can shift the data to the right by 2 bits, that is, the data is reduced by 4 times, and the obtained target data is less than or equal to N1. The selector switch 15 outputs the data transmitted from the shifters 17 based on the third enable signal en3. As such, the data is reduced by 4 times after the first mapping, and the corresponding weight of the target data corresponding to the data is 4.

S103, multiplier and accumulation operation is performed on the target data with the weight of 1 according to a first rule to obtain a first operation value; and multiplier and accumulation operation is performed on the target data with the weight not equal to 1 according to a second rule of multiplying a same column by a same value to obtain a second operation value.

In the embodiment, the first rule is a rule of multiplying a same column by different values. A preset first array circuit 21 can perform the multiplier and accumulation operation on the target data according to the first rule to obtain the first operation value. If the weight corresponding to the target data output in FIG. 2 is 1, or the corresponding enable signal is en1, then the target data is transmitted to the first array circuit 21 for the multiplier and accumulation operation to obtain the corresponding first operation value.

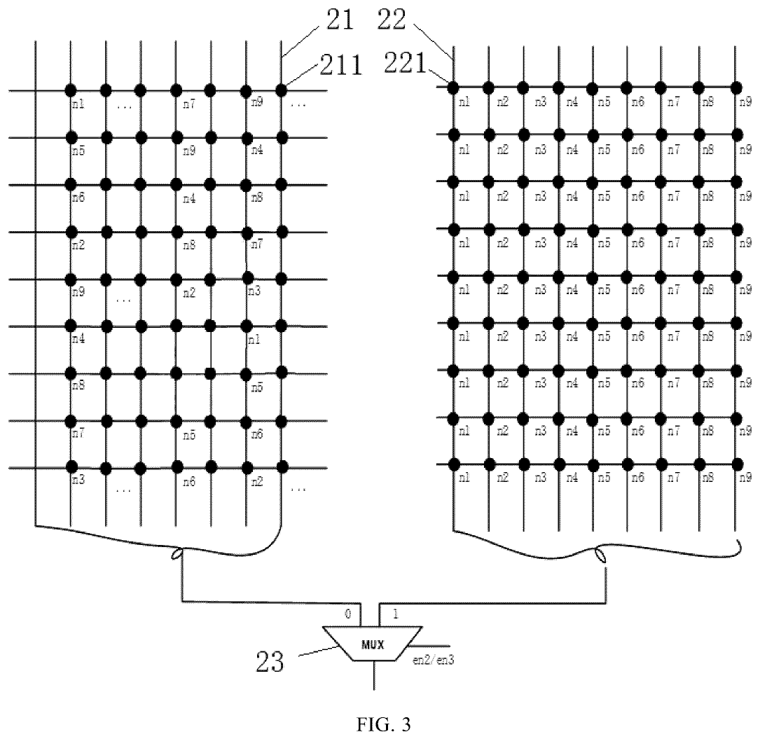

During specific implementation, as shown in FIG. 3, the first array circuit 21 is the same as conventional array circuits, and each cross point 211 in the same column of the first array circuit 21 corresponds to a different value. For example, the values corresponding to the cross points from top to bottom in the left first column of the first array circuit 21 are n1, n5, n6, n2, n9, n4, n8, n7, and n3, respectively. As such, the first operation value can maintain the accuracy of the conversion.

A second array circuit 22 can be used to perform the multiplier and accumulation operation on the target data with the weight not equal to 1 to obtain the second operation value. If the weight corresponding to the target data output in FIG. 2 is not 1, or the corresponding enable signal is en2 or en3, then the target data is transmitted to the second array circuit 22 for the multiplier and accumulation operation to obtain the corresponding second operation value. The second array circuit 22 is different from conventional array circuits, and each cross point 221 in the same column of the second array circuit 22 corresponds to a same value. For example, the value corresponding to each cross point in the left first column of the second array circuit 22 is n1. As such, the second operation value remains linear, which can restore a second quantized value subsequently corresponding to the second operation value.

The first array circuit 21 and the second array circuit 22 can output data selectively through a selector switch 23. When the selector switch 23 receives the second enable signal en2 or the third enable signal en3, the result (second operation value) of the second array circuit 22 is output. When the selector switch 23 does not receive the second enable signal en2 or the third enable signal en3 or receives the first enable signal en1, the result (first operation value) of the first array circuit 21 is output.

In this example, the values corresponding to the cross points in the first array circuit 21 and the second array circuit 22 (i.e., n1, n2, n3, etc.) represent weights of a neural network. The neural network can calculate the weights and optimize the weights as needed.

S104, analog-to-digital conversion is performed on the first operation value and the second operation value to obtain a first quantized value and a second quantized value, respectively.

In the embodiment, after the first operation value and the second operation value are calculated, an analog-to-digital conversion module can be used to perform the analog-to-digital conversion on the first operation value and the second operation value separately, so that the first operation value and the second operation value are converted into digital signals, and the corresponding first quantized value and second quantized value are obtained.

S105, second mapping is performed on the first quantized value and the second quantized value based on the weights to obtain a third quantized value and a fourth quantized value, and the second mapping is reverse to the first mapping.

In the embodiment, the second mapping is reverse to the first mapping. For example, the first mapping is about reducing the data by a multiple of 2, and the second mapping is about expanding the data by a multiple of 2.

In the embodiment, since the weight of the target data corresponding to the first quantized value is 1 (i.e., the corresponding first mapping is about reducing by a multiple of 1), the second mapping performed on the first quantized value is about expanding by a multiple of 1. Therefore, it can be determined that the third quantized value obtained after the second mapping on the first quantized value is the same as the first quantized value. For the second quantized value, the fourth quantized value can be obtained by expanding by a corresponding multiple based on the weight of the target data corresponding to the second quantized value.

In some embodiments, the performing the second mapping on the first quantized value and the second quantized value based on the weights to obtain the third quantized value and the fourth quantized value, the second mapping being reverse to the first mapping, may include:
S1051, multiplying the weight of the target data corresponding to the first quantized value by the first quantized value to obtain the third quantized value; and
S1052, multiplying the weight of the target data corresponding to the second quantized value by the second quantized value to obtain the fourth quantized value.

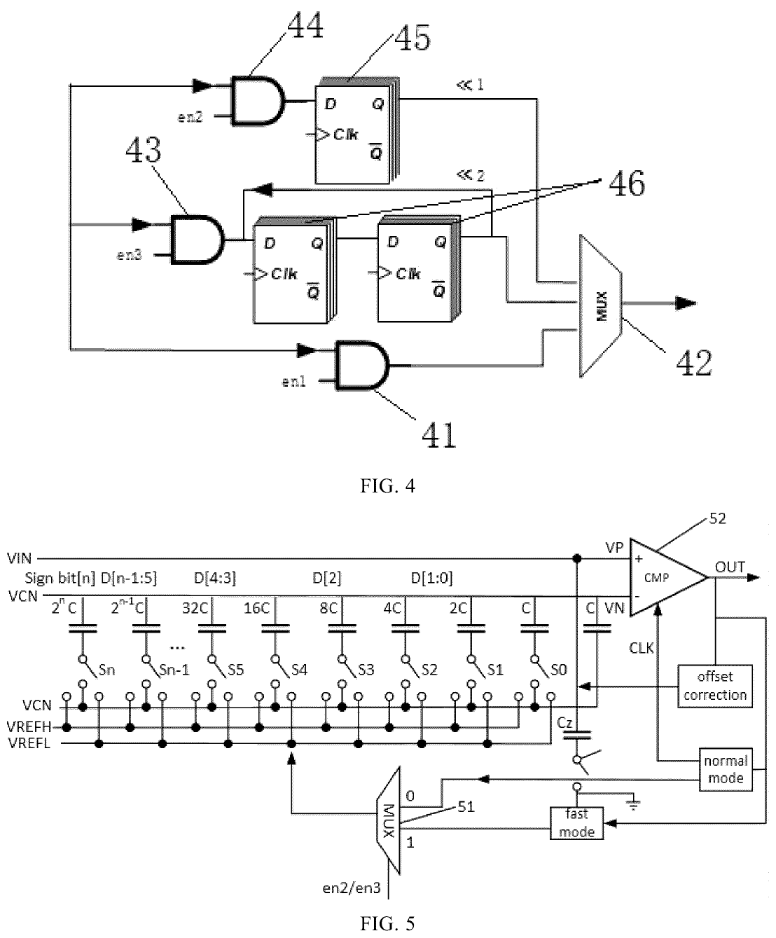

During specific implementation, a second mapping module shown in FIG. 4 can be used to perform the second mapping on the first quantized value and the second quantized value. The first quantized value and the second quantized value can be input to an input terminal/end of the second mapping module separately. The weight of the target data corresponding to the first quantized value is 1, corresponding to the first enable signal en1, and the first enable signal en1 is transmitted to a switch 41 in the second mapping module; when the second mapping module processes the first quantized value, the first quantized value is transmitted to a selector switch 42 through the switch 41 in the second mapping module under the enable of the first enable signal en1, and the selector switch 42 outputs the first quantized value (i.e., the third quantized value) transmitted from the switch 41. As such, the first quantized value is maintained unchanged after the second mapping. The weight of the target data corresponding to the second quantized value is not 1, corresponding to the second enable signal en2 or the third enable signal en3. The second enable signal en2 is transmitted to a switch 44, and the third enable signal is transmitted to a switch 43. The switch 44 is connected to the selector switch 42 through a shifter 45, and the shifter 45 can shift the second quantized value to the left by 1 bit, that is, expand the second quantized value by a multiple of 2. The switch 43 is connected to the selector switch 42 by shifters 46 (including two shifters), and the shifters 46 can shift the second quantized value to the left by 2 bits, that is, expand the second quantized value by a multiple of 4.

When the second mapping module processes the second quantized value, the second quantized value is transmitted to the selector switch 42 through the switch 44 in the second mapping module under the enable of the second enable signal en2, and the selector switch 42 outputs the fourth quantized value transmitted from the shifter 45. As such, the second quantized value is expanded by a multiple of 2 after the second mapping. The second quantized value corresponding to the second operation value output by the second array circuit 22 is restored.

When the second mapping module processes the second quantized value, the second quantized value is output to the selector switch 42 through the switch 43 in the second mapping module under the enable of the third enable signal en3, and the selector switch 42 selects to output the data (the fourth quantized value) transmitted from the shifters 46. As such, the second quantized value is expanded by a multiple of 4 after the second mapping. The second quantized value corresponding to the second operation value output by the second array circuit 22 is restored.

S106, the third quantized value and the fourth quantized value is summed to obtain a target output value.

In the embodiment, the target data in the target array data is divided into two parts for processing, so that the third quantized values corresponding to one part of the target data and the fourth quantized values corresponding to the other part of the target data are added before the data is output, which can restore the finally output data and ensure the accuracy of the output data.

According to the data processing method provided in the embodiments of the present application, the first mapping is performed on the to-be-processed array data to obtain the target array data, and the weight corresponding to each target data in the target array data during the first mapping is determined; where the first mapping is about reducing the data in the to-be-processed array data, and the reduced data all belongs to the first numerical range, so that the numerical range of the target data in the target array data is less than that of the data in the to-be-processed array data. As such, the numerical range of the to-be-processed array data can be effectively converged, making the input data of the multiplier and accumulation operation process (that is, the numerical range of the target array data) smaller, thereby reducing computational power consumption of array circuits. In addition, the multiplier and accumulation operation is performed on the target data with the weight of 1 according to the first rule to obtain the first operation value, and the multiplier and accumulation operation is performed on the target data with the weight not equal to 1 according to the second rule of multiplying the same column by the same value to obtain the second operation value, thereby the second operation value can be maintained linear, and the first operation value maintains conversion accuracy. On the one hand, the conversion accuracy loss of overall operation values is reduced, and on the other hand, the second quantized value subsequently corresponding to the second operation value can be restored. Then, the analog-to-digital conversion is performed on the first operation value and the second operation value separately to obtain the first quantized value and the second quantized value, and the first operation value and the second operation value have smaller numerical ranges, which can improve the conversion precision of an analog-to-digital conversion circuit and shorten the conversion time. Next, the second mapping is performed on the first quantized value and the second quantized value based on the weights to obtain the third quantized value and the fourth quantized value, and the second mapping is reverse to the first mapping, thereby achieving the restoration of the second quantized value corresponding to the second operation value. Finally, the third quantized value and the fourth quantized value are summed to obtain the target output value, thereby achieving the restoration of the input data.

In an optional embodiment, the step 104 of performing the analog-to-digital conversion on the first operation value and the second operation value separately to obtain the first quantized value and the second quantized value includes:
S1041, calculating, by an analog-to-digital converter, a first difference between the first operation value and a reference value and a second difference between the second operation value and the reference value, respectively;
S1042, when absolute values of the first and second differences are less than a threshold, sampling analog signals output from different bits of the analog-to-digital converter in order from low to high bits; and
S1043, determining the first quantized value based on a bit value of the analog-to-digital converter corresponding to the analog signal matching the first operation value, and determining the second quantized value based on a bit value of the analog-to-digital converter corresponding to the analog signal matching the second operation value.

In the embodiment, by comparing the difference between the first operation value and the reference value and the difference between the second operation value and the reference value, the analog signals output from different bits of the analog-to-digital converter can be sampled in order from low to high bits when the absolute values of the differences are less than the threshold, which can greatly reduce the analog-to-digital conversion time and also reduce the conversion power.

In one implementation, the calculating the second difference between the second operation value and the reference value by the analog-to-digital converter includes: determining a correction value corresponding to the second operation value by the analog-to-digital converter based on the weight of the target data corresponding to the second operation value; calculating a third difference between the second operation value and the correction value; and designating a difference between the third difference and the reference value as the second difference between the second operation value and the reference value.

During specific implementation, an analog-to-digital converter as shown in FIG. 5 can be used to perform the analog-to-digital conversion on the first operation value and the second operation value. The analog-to-digital converter includes a normal mode and a fast mode. In the normal mode, the analog-to-digital converter samples analog signals output from its different bits (including Sign bit0-n bits) in order from high to low bits. In the fast mode, the analog-to-digital converter samples analog signals output from its different bits in order from low to high bits. The reference value is represented by VCM, VIN represents the first or second operation value, and a Cz capacitor is connected in series to an input terminal. When a selector switch 51 in the analog-to-digital converter receives the second enable signal en2 or the third enable signal en3, the fast mode is activated. In the fast mode, Vp is further converted into VIN−VCz (i.e., the difference between the second operation value and the correction value), VIN represents the second operation value, VCz represents the correction value corresponding to the second operation value, and Vp represents the operation value input to a comparator 52. As such, the conversion efficiency of the analog-to-digital converter can be further improved.

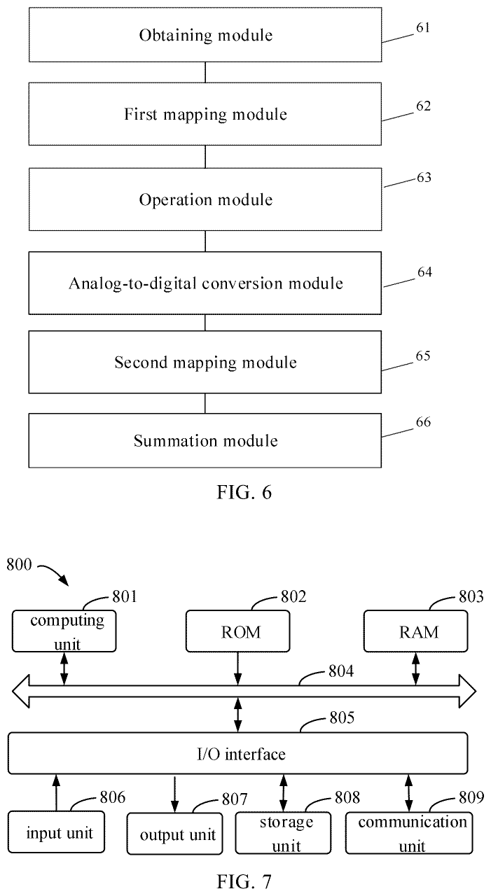

An embodiment of the present application further provides a data processing apparatus, applied to a CIM circuit, as shown in FIG. 6, which includes:
an obtaining module 61, configured to obtain to-be-processed array data and transmit the same to a first mapping module 62;
the first mapping module 62, configured to perform first mapping on the to-be-processed array data to obtain target array data, and determine a weight corresponding to each target data in the target array data during the first mapping; where a numerical range of the target data in the target array data is less than that of data in the to-be-processed array data; the first mapping module 62 is the first mapping module shown in FIG. 2;
an operation module 63, configured to perform multiplier and accumulation operation on the target data with the weight of 1 according to a first rule to obtain a first operation value; and perform multiplier and accumulation operation on the target data with the weight not equal to 1 according to a second rule of multiplying a same column by a same value to obtain a second operation value; the operation module 63 is the module shown in FIG. 3, including a first array circuit 21, a second array circuit 22, and a selector switch 23;
an analog-to-digital conversion module 64, configured to perform analog-to-digital conversion on the first operation value and the second operation value to obtain a first quantized value and a second quantized value, respectively; the analog-to-digital conversion module 64 is the analog-to-digital converter shown in FIG. 5;
a second mapping module 65, configured to perform second mapping on the first quantized value and the second quantized value based on the weights to obtain a third quantized value and a fourth quantized value; where the second mapping is reverse to the first mapping; the second mapping module 65 is the second mapping module shown in FIG. 4; and
a summation module 66, configured to sum the third quantized value and the fourth quantized value to obtain a target output value.

In an optional embodiment, the first mapping module 62 is configured to: determine a numerical range corresponding to each data in the to-be-processed array data; perform the first mapping on each data based on the numerical range corresponding to each data to maintain the data belonging to a first numerical range unchanged and map the data greater than the first numerical range to data of the first numerical range to obtain the target array data, and determine a weight of each data during the first mapping as the weight corresponding to each target data in the target array data.

According to the data processing apparatus provided in the embodiments of the present application, the first mapping is performed on the to-be-processed array data to obtain the target array data, and the weight corresponding to each target data in the target array data during the first mapping is determined; where the numerical range of the target data in the target array data is less than that of the data in the to-be-processed array data. As such, the numerical range of the to-be-processed array data can be effectively converged, making the input data of the multiplier and accumulation operation process (that is, the numerical range of the target array data) smaller, thereby reducing computational power consumption of array circuits. In addition, the multiplier and accumulation operation is performed on the target data with the weight of 1 according to the first rule to obtain the first operation value, and the multiplier and accumulation operation is performed on the target data with the weight not equal to 1 according to the second rule of multiplying the same column by the same value to obtain the second operation value, thereby the second operation value can be maintained linear, and the first operation value maintains conversion accuracy. On the one hand, the conversion accuracy loss of overall operation values is reduced, and on the other hand, the second quantized value subsequently corresponding to the second operation value can be restored. Then, the analog-to-digital conversion is performed on the first operation value and the second operation value separately to obtain the first quantized value and the second quantized value, and the first operation value and the second operation value have smaller numerical ranges, which can improve the conversion precision and time of an analog-to-digital conversion circuit. Next, the second mapping is performed on the first quantized value and the second quantized value based on the weights to obtain the third quantized value and the fourth quantized value, and the second mapping is reverse to the first mapping, thereby achieving the restoration of the second quantized value corresponding to the second operation value. Finally, the third quantized value and the fourth quantized value are summed to obtain the target output value, thereby achieving the restoration of the output data.

According to embodiments of the present application, the present application further provides an electronic device and a readable storage medium.

FIG. 7 illustrates a schematic block diagram of an exemplary electronic device 800 that can be used to implement the embodiments of the present application. The electronic device is intended to represent various forms of digital computers, such as a laptop computer, a desktop computer, a workstation, a personal digital assistant, a server, a blade server, a mainframe computer, and other suitable computers. The electronic device may further represent various forms of mobile apparatuses, such as a personal digital assistant, a cellular phone, a smart phone, a wearable device, and other similar computing apparatuses. The components shown herein, their connections and relationships and their functions are merely examples, and are not intended to limit the implementation of the present application described and/or required herein.

As shown in FIG. 7, the device 800 includes a computing unit 801, which may perform various appropriate actions and processing according to a computer program stored in a read-only memory (ROM) 802 or a computer program loaded from a storage unit 808 to a random access memory (RAM) 803. The RAM 803 may further store various programs and data required for the operation of the device 800. The computing unit 801, the ROM 802, and the RAM 803 are connected to each other through a bus 804. An input/output (I/O) interface 805 is also connected to the bus 804.

A plurality of components in the device 800 are connected to the I/O interface 805, including: an input unit 806, such as a keyboard, a mouse and the like; an output unit 807, such as various types of displays, speakers and the like; the storage unit 808, such as a magnetic disk, an optical disk and the like; and a communication unit 809, such as a network card, a modem, a wireless communication transceiver and the like. The communication unit 809 allows the device 800 to exchange information/data with other devices over a computer network such as the Internet and/or various telecommunication networks.

The computing unit 801 may be various general-purpose and/or special-purpose processing components with processing and computing capabilities. Some examples of the computing unit 801 include, but are not limited to, a central processing unit (CPU), a graphics processing unit (GPU), various dedicated artificial intelligence (AI) computing chips, various computing units that run machine learning model algorithms, a digital signal processor (DSP), and any appropriate processor, controller, microcontroller and the like. The computing unit 801 performs various methods and processing described above, such as the data processing method. For example, in some embodiments, the data processing method may be implemented as a computer software program, which is tangibly included in a machine-readable medium, such as the storage unit 808. In some embodiments, a part or all of the computer program may be loaded and/or installed onto the device 800 via the ROM 802 and/or the communication unit 809. When the computer program is loaded onto the RAM 803 and executed by the computing unit 801, one or more steps of the data processing method described above can be performed. Alternatively, in other embodiments, the computing unit 801 may be configured, by any other suitable means (for example, by means of firmware), to perform the data processing method.

Various implementations of the systems and technologies described herein above can be implemented in a digital electronic circuit system, an integrated circuit system, a field programmable gate array (FPGA), an application-specific integrated circuit (ASIC), an application-specific standard product (ASSP), a system-on-chip (SOC) system, a complex programmable logical device (CPLD), computer hardware, firmware, software, and/or a combination thereof. The implementations may include: being implemented in one or more computer programs, the one or more computer programs may be executed and/or interpreted in a programmable system including at least one programmable processor, and the programmable processor may be a dedicated or general-purpose programmable processor, and may receive data and instructions from a storage system, at least one input apparatus, and at least one output apparatus, and transmit the data and instructions to the storage system, the at least one input apparatus, and the at least one output apparatus.

Program codes used to perform the method of the present application can be written in any combination of one or more programming languages. These program codes may be provided for a processor or a controller of a general-purpose computer, a special-purpose computer, or other programmable data processing apparatuses, such that when the program codes are executed by the processor or the controller, the functions/operations specified in the flowcharts and/or block diagrams are implemented. The program codes may be completely executed on a machine, or partially executed on a machine, or may be, as an independent software package, partially executed on a machine and partially executed on a remote machine, or completely executed on a remote machine or a server.

In the context of the present application, the machine-readable medium may be a tangible medium, which may include or store a program for use by an instruction execution system, apparatus or device, or for use in combination with the instruction execution system, apparatus or device. The machine-readable medium may be a machine-readable signal medium or a machine-readable storage medium. The machine-readable medium may include, but is not limited to, an electronic, magnetic, optical, electromagnetic, infrared, or semiconductor system, apparatus, or device, or any suitable combination thereof. More specific examples of the machine-readable storage medium may include an electrical connection based on one or more wires, a portable computer disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof.

To provide interaction with a user, the systems and technologies described herein may be implemented in a computer, the computer is provided with: a display apparatus (such as a cathode ray tube (CRT) or a liquid crystal display (LCD) monitor) used for displaying information to the user; and a keyboard and a pointing apparatus (such as a mouse or a trackball), and the user may provide input to the computer through the keyboard and the pointing apparatus. Other types of apparatuses may also be used for providing interaction with the user; for example, feedback provided to the user may be sensory feedback in any form (such as visual feedback, auditory feedback, or tactile feedback); and the input of the user may be received in any form (including vocal input, speech input, or tactile input).

The systems and technologies described herein may be implemented in a computing system (for example, as a data server) including a background component, or a computing system (for example, an application server) including a middleware component, or a computing system (for example, a user computer with a graphical user interface or a web browser through which the user may interact with the implementation manners of the systems and technologies described herein) including a front-end component, or a computing system including any combination of the background component, the middleware component, or the front-end component. The components of the system may be connected with each other through digital data communication (for example, a communication network) in any form or medium. Examples of the communication network include: a local area network (LAN), a wide area network (WAN), and the Internet.

The computer system may include a client and a server. The client and the server are generally far away from each other and usually interact through the communications network. A relationship between the client and the server is generated by computer programs running in respective computers and having a client-server relationship with each other. The server may be a cloud server, a server in a distributed system, or a server combined with a blockchain.

It should be understood that the steps may be reordered, added or deleted by using the flows in various forms, which are shown above. For example, the steps recorded in the present application may be performed concurrently, in order, or in a different order, provided that the desired results of the technical solutions disclosed in the present application can be achieved, which is not limited herein.

In addition, the terms “first” and “second” are merely used for a description purpose, and cannot be interpreted as indicating or implying relative importance or implicitly indicating the number of the technical features indicated. Therefore, the features defined by “first” and “second” can explicitly or implicitly include at least one of the features. In the description of the present application, “a plurality of” means two or more, unless otherwise specified.

The above merely describes specific embodiments of the present application, but the protection scope of the present application is not limited thereto. Any person skilled in the art can easily conceive modifications or replacements within the technical scope of the present application, and these modifications or replacements shall fall within the protection scope of the present application. Therefore, the protection scope of the present application shall be subject to the protection scope of the claims.

1. A data processing method, comprising:
obtaining to-be-processed array data;
performing first mapping on the to-be-processed array data to obtain target array data, and determining a weight corresponding to each target data in the target array data during the first mapping; wherein a numerical range of the target data in the target array data is less than a numerical range of data in the to-be-processed array data;
performing multiplier and accumulation operation on the target data with the weight of 1 according to a first rule to obtain a first operation value; and performing the multiplier and accumulation operation on the target data with the weight not equal to 1 according to a second rule of multiplying a same column by a same value to obtain a second operation value;
performing analog-to-digital conversion on the first operation value and the second operation value to obtain a first quantized value and a second quantized value, respectively;
performing second mapping on the first quantized value and the second quantized value based on the weights to obtain a third quantized value and a fourth quantized value; wherein the second mapping is reverse to the first mapping; and
summing the third quantized value and the fourth quantized value to obtain a target output value.

2. The data processing method according to claim 1, wherein the step of performing the first mapping on the to-be-processed array data to obtain the target array data, and determining the weight corresponding to each target data in the target array data during the first mapping, the numerical range of the target data in the target array data being less than the numerical range of the data in the to-be-processed array data, comprises:
determining a numerical range corresponding to each data in the to-be-processed array data; and
performing the first mapping on each data based on the numerical range corresponding to each data to maintain the data belonging to a first numerical range unchanged and map the data greater than the first numerical range to data of the first numerical range to obtain the target array data, and determining a weight of each data during the first mapping as the weight corresponding to each target data in the target array data during the first mapping.

3. The data processing method according to claim 2, wherein the step of performing the first mapping on each data based on the numerical range corresponding to each data to maintain the data belonging to the first numerical range unchanged and map the data greater than the first numerical range to the data of the first numerical range to obtain the target array data, and determining the weight of each data during the first mapping as the weight corresponding to each target data in the target array data during the first mapping comprises:
when the data belongs to the first numerical range, determining that the data is maintained unchanged during the first mapping and the weight corresponding to the target data obtained after the first mapping on the data is 1;
when the data belongs to a second numerical range, determining that the data is reduced by a first preset multiple during the first mapping and the weight corresponding to the target data obtained after the first mapping on the data is the first preset multiple; wherein the second numerical range is greater than the first numerical range; and
when the data belongs to a third numerical range, determining that the data is reduced by a second preset multiple during the first mapping and the weight corresponding to the target data obtained after the first mapping on the data is the second preset multiple; wherein the third numerical range is greater than the second numerical range, and the second preset multiple is greater than the first preset multiple.

4. The data processing method according to claim 1, wherein the step of performing the analog-to-digital conversion on the first operation value and the second operation value to obtain the first quantized value and the second quantized value respectively comprises:
calculating, by an analog-to-digital converter, a first difference between the first operation value and a reference value and a second difference between the second operation value and the reference value, respectively;
when absolute values of the first and second differences are less than a threshold, sampling analog signals output from different bits of the analog-to-digital converter in order from low to high bits; and
determining the first quantized value based on a bit value of the analog-to-digital converter corresponding to the analog signal matching the first operation value, and determining the second quantized value based on a bit value of the analog-to-digital converter corresponding to the analog signal matching the second operation value.

5. The data processing method according to claim 4, wherein the step of calculating the second difference between the second operation value and the reference value by the analog-to-digital converter comprises:
determining a correction value corresponding to the second operation value by the analog-to-digital converter based on the weight of the target data corresponding to the second operation value;
calculating a third difference between the second operation value and the correction value; and
designating a difference between the third difference and the reference value as the second difference between the second operation value and the reference value.

6. The data processing method according to claim 1, wherein the step of performing the second mapping on the first quantized value and the second quantized value based on the weights to obtain the third quantized value and the fourth quantized value, the second mapping being reverse to the first mapping, comprises:
multiplying the weight of the target data corresponding to the first quantized value by the first quantized value to obtain the third quantized value; and
multiplying the weight of the target data corresponding to the second quantized value by the second quantized value to obtain the fourth quantized value.

7. A data processing apparatus, comprising:
an obtaining module, configured to obtain to-be-processed array data;
a first mapping module, configured to perform first mapping on the to-be-processed array data to obtain target array data, and determine a weight corresponding to each target data in the target array data during the first mapping; wherein a numerical range of the target data in the target array data is less than a numerical range of data in the to-be-processed array data;
an operation module, configured to perform multiplier and accumulation operation on the target data with the weight of 1 according to a first rule to obtain a first operation value; and
perform the multiplier and accumulation operation on the target data with the weight not equal to 1 according to a second rule of multiplying a same column by a same value to obtain a second operation value;
an analog-to-digital conversion module, configured to perform analog-to-digital conversion on the first operation value and the second operation value to obtain a first quantized value and a second quantized value, respectively;
a second mapping module, configured to perform second mapping on the first quantized value and the second quantized value based on the weights to obtain a third quantized value and a fourth quantized value; wherein the second mapping is reverse to the first mapping; and
a summation module, configured to sum the third quantized value and the fourth quantized value to obtain a target output value.

8. The data processing apparatus according to claim 7, wherein the first mapping module is configured to:
determine a numerical range corresponding to each data in the to-be-processed array data; and
perform the first mapping on each data based on the numerical range corresponding to each data to maintain the data belonging to a first numerical range unchanged and map the data greater than the first numerical range to data of the first numerical range to obtain the target array data; and determine a weight of each data during the first mapping as the weight corresponding to each target data in the target array data during the first mapping.

9. An electronic device, comprising:
at least one processor; and
a memory in communication connection with the at least one processor;
wherein the memory stores instructions executable by the at least one processor, and the instructions are executed by the at least one processor to cause the at least one processor to execute the data processing method according to claim 1.

10. A non-transitory computer-readable storage medium having stored therein computer instructions that, when executed by a computer, cause the computer to perform the data processing method according to claim 1.
